(19) United States
(12) Patent Application Publication
(10) Pub. No.: US 2015/0018630 A1
(43) Pub. Date: Jan. 15, 2015

(54) SYSTEMS AND METHODS FOR CREATING COMPREHENSIVE AND PERSONALIZED BRAIN HEALTH PROGRAMS

(71) Applicant: NEUREXPAND BRAIN CENTER LLC, Lutherville, MD (US)

(72) Inventor: Majid FOTUHI, Lutherville, MD (US)

(21) Appl. No.: 14/332,340

(22) Filed: Jul. 15, 2014

Related U.S. Application Data
(60) Provisional application No. 61/846,578, filed on Jul. 15, 2013.

(51) Int. Cl. A61B 5/00; A61B 8/08; A61B 5/0205

(52) U.S. Cl. CPC A61B 5/4064; A61B 5/0205; A61B 8/488; A61B 5/055
USPC 600/300

(57) ABSTRACT

Systems, methods and computer-readable storage media for generating comprehensive and personalized brain health programs are described. The brain health programs may be configured to improve the brain health of a patient, including improving patient cognitive function and/or increasing hippocampal volume. The brain health programs may use various interventions, including, without limitation, exercises, activities, habits, and diet, that are directed toward certain aspects of a patient's brain and/or brain function, such as memory abilities or increasing the size of the hippocampus or other parts of the brain.

### CROSS-REFERENCE TO RELATED APPLICATIONS

This application claims the benefit of U.S. Provisional Application No. 61/846,578 filed on Jul. 15, 2013, the contents of which are incorporated by reference in their entirety as if fully set forth herein.

### BACKGROUND

Individuals beyond the age of 80 represent the most rapidly growing segment of the United States population. This segment is prone to cognitive decline and Alzheimer's disease. In addition, millions of middle-aged individuals are experiencing health conditions such as hypertension, sleep apnea, obesity, concussion, sedentary lifestyle, depression, and diabetes mellitus. These risk factors have a negative impact on their brain and lead to memory loss and cognitive deficits. Accordingly, cognitive decline, and particularly cognitive decline with aging, has become a major public health issue in the United States and worldwide.

Brain health may be impacted by genetic risk factors for Alzheimer's disease plus a wide range of other conditions many of which can be treated. However, conventional treatments for cognitive decline focus on one condition or etiology at a time and often disregard other possible causes. For example, a treatment may focus on diet and exercise, but may ignore cognitive training. As such, a patient may be required to visit multiple healthcare providers and seek several varied courses of treatment to address even a few possible causes of cognitive decline. Accordingly, it would be beneficial to have a multidisciplinary personalized treatment program for treatment of all of the unique brain conditions that may cause cognitive decline and memory loss of a patient in an efficient and comprehensive manner.

### SUMMARY

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

In an embodiment, a method for generating at least one comprehensive and personalized brain health program for a patient may comprise receiving health information associated with the patient, receiving diagnostic testing information associated with the patient, generating a brain health assessment for the patient based on at least a portion of the health information and the diagnostic testing information, the brain health assessment comprising a plurality of brain health conditions, generating the at least one comprehensive and personalized brain health program for the patient based on the at least one assessment, the at least one comprehensive and personalized brain health program comprising a plurality of interventions configured to improve or maintain the plurality of brain health conditions.

In an embodiment, a system for generating at least one comprehensive and personalized brain health program for a patient may comprise a processor and a non-transitory, computer-readable storage medium in operable communication with the processor. The computer-readable storage medium may contain one or more programming instructions that, when executed, cause the processor to: receive health information from the patient, receive diagnostic testing information associated with the patient, generate a brain health assessment for the patient based on at least a portion of the health information and the diagnostic testing information, the brain health assessment comprising a plurality of brain health conditions, and generate at least one comprehensive and personalized brain health program for the patient based on the at least one assessment, the at least one comprehensive and personalized brain health program comprising a plurality of interventions configured to improve or maintain the plurality of brain health conditions.

In an embodiment, a computer-readable storage medium may have computer-readable program code configured to generate at least one comprehensive and personalized brain health program embodied therewith. The computer-readable program code may include computer-readable program code configured to receive health information associated with the patient, computer-readable program code configured to receive diagnostic testing information associated with the patient, computer-readable program code configured to generate a brain health assessment for the patient based on at least a portion of the health information and the diagnostic testing information, the brain health assessment comprising a plurality of brain health conditions, and computer-readable program code configured to generate the at least one comprehensive and personalized brain health program for the patient based on the at least one assessment, the at least one comprehensive and personalized brain health program comprising a plurality of interventions configured to improve or maintain the plurality of brain health conditions.

### BRIEF DESCRIPTION OF THE DRAWINGS

The above and other objects of the present invention will become more readily apparent from the following detailed description taken in connection with the accompanying drawings.

FIG. 1A depicts illustrative conditions that may affect brain function.

FIG. 1B depicts illustrative categories of brain interventions according to some embodiments.

FIG. 2 depicts an illustrative method for generating a brain health program according to some embodiments.

FIG. 3 depicts an illustrative system for generating a brain health program according to some embodiments.

FIG. 4 depicts an illustrative method for generating a brain health program according to an embodiment.

FIGS. 5-8 graphically depict various aspects of an example study of patients participating in comprehensive and personalized brain health programs.

FIG. 9 illustrates various embodiments of a computing device for implementing the various methods and processes described herein

### DETAILED DESCRIPTION

The described technology generally involves methods and systems for generating comprehensive and personalized brain health programs (“brain health programs”) for patients. In general, the brain health programs may be configured to improve the brain health (“brain fitness,” “brain wellness” or “brain health”) of the patient by improving brain conditions associated with the patient, such as memory, cognition, hippocampal atrophy, or the like. The brain health programs may use interventions (for instance, exercises, activities, habits, diet, or the like) directed toward certain aspects of a patient's brain and/or brain function, such as memory abilities or increasing the size of the hippocampus or other parts of the brain.

According to some embodiments, the brain health programs may be formed based on one or more assessments generated using patient health information. In an embodiment, the brain health programs may be comprehensive in that they use multiple interventions concurrently to address the entire brain health of a patient. In some embodiments, the multiple interventions may be used concurrently or substantially concurrently. In an embodiment, the brain health programs may be personalized in that an individualized program is generated for each patient based on her specific brain health and medical information.

Conventional methods for addressing patient brain conditions typically only address one particular need using one single method or activity at a time. As such, even if the treatment is successful, any remaining brain conditions remain wholly or partially untreated. The patient may have to see multiple doctors for multiple conditions impacting their brain conditions and may have to try to maintain numerous, disparate courses of treatment. In addition, each doctor may not be aware of what the other doctors are prescribing or recommending for the patient. As such, one method of treatment may be negatively affecting another course of treatment. For instance, a first doctor may prescribe a medication that may negatively affect the sleeping pattern of the patient, while a second doctor may be counseling the patient in sleep optimization techniques. In addition, conventional methods are generally not individualized based on the patient's particular health history and/or medical information, particularly using all available brain health information for the patient. Accordingly, a patient may not receive the optimum treatment for their unique medical condition and/or collection of conditions.

Brain health programs generated according to some embodiments described herein provide multiple technological advantages. A non-limiting example of a technological advantage is that the brain health programs provide a comprehensive program for improving the brain health of a patient by simultaneously or substantially simultaneously using a constellation of available interventions that will improve brain health. Another non-limiting example of a technological advantage is the brain health programs may be personalized for each patient and their particular brain health needs and conditions, including how these may uniquely interact with each other. In this manner, the brain health programs may provide methods and systems of treating the brain health conditions of a patient that are more effective, efficient and safe than those offered according to conventional technology. In addition, patient monitoring and assessments may be conducted within the shortest time possible and at the same healthcare facility, and reviewed by a single practitioner and/or team that is also the practitioner and/or team that oversees the personal brain health program and follow-up. As such, contrary to conventional techniques, the brain health programs may be performed as efficiently as possible by someone who has knowledge of the entire patient history, instead of being dragged out over a prolonged period of time at multiple facilities and medical personnel.

The brain performs many different functions, including speech, memory and problem solving that are vital to everyday life. Each of these functions may be affected by various aspects of a person's overall brain health, including the size of various parts of the brain and/or the number or type of connections between individual brain cells and between different parts of the brain. The brain also demonstrates neuroplasticity, which is the ability to change. For example, parts of the brain can shrink and/or lose or weaken connections. However, throughout a person's lifespan, the brain also has the ability to grow, getting thicker, denser and larger. Growth of various parts of the brain, which may be achieved according to the brain health programs described according to embodiments provided herein, has many benefits. For example, in the hippocampus, growth may improve short-term memory. Larger frontal lobes have been associated with improved decision making and processing speed. A larger network of connectivity has been shown to improve creativity and the ability to solve abstract puzzles. In general, a bigger brain may enhances brain performance in at least three key areas: memory, clarity and creativity.

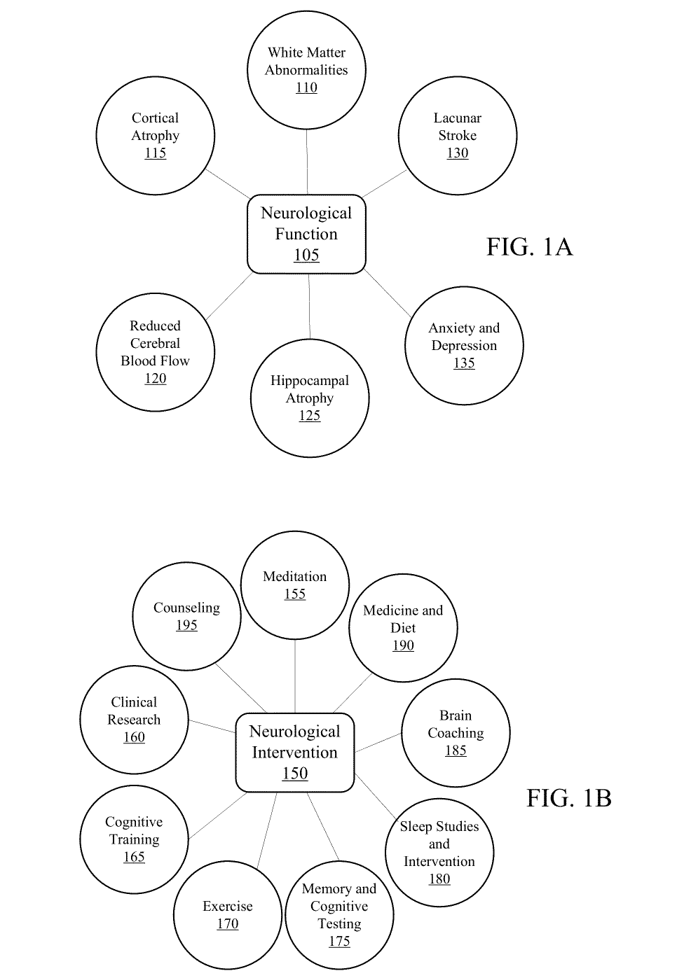

FIG. 1A depicts illustrative conditions that may affect brain function, such as memory recall or problem solving abilities. As shown in FIG. 1A, brain function 105 may be affected by white matter abnormalities 110, such as hypertension (high blood cholesterol and hypertension may indicate that the brain is not getting optimal oxygen flow), diabetes mellitus, congestive heart failure, kidney disease, liver disease, thyroid disease, and/or various vitamin deficiencies, such as Vitamin B₁₂. In addition, white matter abnormalities 110 may be due to aging. For instance, white matter, the network of connections between neurons, may be reduced for certain age groups, primarily due to reduced blood flow

Cortical atrophy 115 may be caused by plaques and tangles, Lewy body disease/synucleopathy, white matter abnormalities 110, stroke, hypoxia, hypertension, diabetes mellitus, congestive heart failure, obstructive sleep apnea, and/or traumatic brain injury. Reduced cerebral blood flow 120 may be caused by hypertension, diabetes mellitus, high cholesterol, smoking, and/or cerebral amyloid angiopathy.

The brain function 105 of a patient may be impacted by the size of the hippocampus, which has some of the greatest potential for neuroplasticity in the human brain. In general, an increased hippocampus leads to improvement in certain brain functions, such as learning and memory, and vice versa. Accordingly, hippocampal atrophy 125 may directly affect the ability of a patient to perform certain important functions. Hippocampal atrophy 125 may occur due to various conditions, including hippocampal sclerosis, obstructive sleep apnea, white matter abnormalities 110, hypoxia, plaques and/or tangles, chronic depression, chronic stress, and/or hypertension. The brain function 105 of a patient may also be impacted by a lacunar stroke 130. The chances of a lacunar stroke 130 may be increased due to various factors, such as, hypertension, diabetes mellitus, high cholesterol, and/or emboli. The brain function 105 of a patient may additionally be affected by stress and/or psychological disorders, such as anxiety and depression 135, which have been shown to negatively impact memory and cognition.

As described in more detail below, brain health programs configured according to some embodiments may use various interventions directed toward the conditions that may impact the brain function of a patient, such as the conditions 110-130 described in FIG. 1A above. FIG. 1B depicts illustrative categories of brain interventions according to some embodiments. As shown in FIG. 1B, meditation training 155 may be a potential category of brain intervention 150, which may also include other activities, such as massage. Participation in clinical research 160 may provide various benefits, including further awareness of the condition and/or information that may be used to treat a condition. Cognitive training 165 may improve cognitive function and other problem solving capabilities of the brain through puzzles, books, and problem solving practice. Exercise 170 and general physical fitness may operate to, among other things, improve mood, memory, cognitive functions, and a larger hippocampus. For example, individuals who regularly burn a high level of calories (for instance, from exercise) may have a higher percentage of grey matter in the frontal, parietal, and temporal lobes, including the hippocampus, compared with individuals who burn fewer calories.

A component of exercise 170 may include improving balance and equilibrium that may operate to prevent injury, particular head injuries, which may be an issue for certain patients (for instance, patients over a certain age). Memory and cognitive testing 175 may be configured to strengthen memory, attention, verbal fluency, and problem solving skills.

Lack of sleep and sleep disorders, such as insomnia and sleep apnea, may lead to increased atrophy of parts of the brain. Accordingly, sleep studies and sleep-related intervention 180 may operate to improve the brain function of a patient. Brain coaching 185 may include a brain health program and progress monitoring to ensure that a program is benefiting the patient. Medicine and diet 190 are a central feature of brain health, for instance, a proper diet may counteract brain atrophy and even facilitate a thicker cortex and a larger hippocampus. As described above, stress and psychological disorders, including, without limitation, anxiety and depression 135, have been shown to negatively impact memory and cognition. Brain coaching 185 may include psycho-education on stress reduction techniques and counseling 195 that can mitigate such stress and psychological disorders to improve patient outcomes.

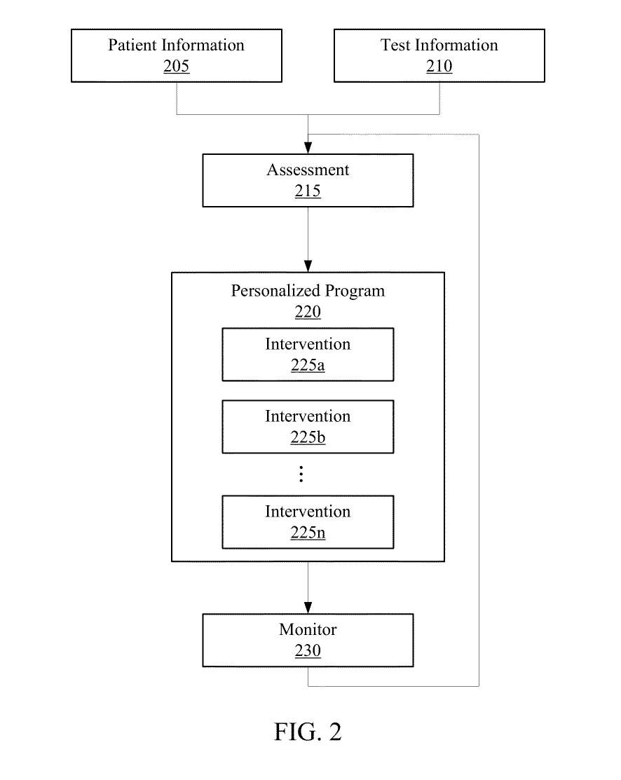

FIG. 2 depicts an illustrative method for generating a brain health program according to some embodiments. As shown in FIG. 1, patient information 205 and test information 210 are used to generate an assessment 215. The patient information 205 may include any information associated with the patient that may be used in generating the assessment 215 and, ultimately, the personalized program 220. Patient information 205 may include, without limitation, medical records, questionnaire answers (for example, questions about cognitive abilities, memory, problem solving, or the like), demographic information, brain health goals (for example, improved memory, reduction in risk of Alzheimer's disease, or the like), and/or any other type of information that the patient may provide for generating the assessment 215. At least a portion of the patient information 210 may be determined at an initial consultation, for instance, with a neurologist or other medical personnel, at the healthcare facility wherein the brain health program may be generated.

The test information 210 may include any information obtained responsive to the patient undergoing medical tests, exams, screenings, or the like. Non-limiting examples of test information 210 may include information from blood tests, brain exams, memory screenings, cognitive screenings, neuropsychological evaluations, physical exams, and MRIs and quantitative MRIs, sleep studies (for instance, a two night study performed at the patient's home or a sleep study facility), cardiopulmonary fitness testing (for instance, a cardiopulmonary exercise test (CPET)), an actigraph (for instance, a five day actigraph of patient activity that may measure insomnia at night and activity during the day), electroencephalography (EEG), and a quantitative EEG (Q-EEG or “brain mapping”), carotid ultrasound, and/or transcranial Doppler (TCD).

The brain exams may include various questions and/or exams directed to determining a brain health baseline for the patient, including memory, hearing, vision, strength, posture, reflexes, coordination, gait, and mood. A memory screen may include the patient answering questions, such as through a touch-screen monitor configured to display questions that measure memory, fluency and higher brain functions. A cognitive evaluation may include a battery of tests configured to assess the patient's cognitive abilities and/or brain strengths and weaknesses. For instance, the cognitive evaluation may determine that a patient has strong short-term memory skills but weak problem-solving skills. In an embodiment, the test may be the same or similar to those used by those having ordinary skill in the art, such as a psychometrician. In an embodiment, the cognitive evaluation may be configured to measure the function of each area of the cortex and hippocampus.

EEG technology may be used to measure electrical activity in different parts of the brain, for instance, to locate any excessively fast or slow brain waves. The EEG information may be used, among other things, to generate a “brain map” of brain activity. The brain map may be used to check brain wave frequency and look for signs of various health conditions, including anxiety, depression, insomnia, and/or Attention Deficit Disorder (ADD). A carotid ultrasound and TCD may be used to examine the amount of blood flow inside of the brain, for example, because adequate cerebral blood flow is an important factor in optimum brain health. The ultrasound may be of the blood vessels in your neck, to check for signs of blockages in the blood flow to the brain. Blood tests may be used to determine levels of elements that may impact brain health, including thyroid hormones, Vitamin B₁₂, Vitamin D, red blood cells (for example, for anemia). Deficiencies in certain elements may lead to fatigue, memory loss and/or difficulty concentrating. A brain MRI may be used to determine whether the patient has had a stroke or other structural abnormalities, as well as to measure atrophy in various parts of the brain, for example, that are important for memory and/or cognition. In addition, signs of brain aging may also be detected through an MRI. For example, aging may be demonstrated as a thinning of the cortex, visible in an MRI of the brain. Stamina and endurance levels may be measured using a CPET configured to measure the maximum volume of oxygen (“VO₂ max”).

In an embodiment, the patient information 205 and/or test information 210 may be compared to the same or similar information from other individuals, for example, to compare the patient with those having similar characteristics, such as age, gender, education, occupation, or the like. In an embodiment, one or more of the tests may be repeated at a later time and compared with earlier results, for instance, to monitor the progress of the patient.

The patient information 205 and/or the test information 210 may be used to generate an assessment 215 of the brain health of the patient. The assessment 215 may be generated by processing all of the available information 205, 210 to determine various aspects of the brain health of the patient including, without limitation, level of brain functions (for instance, memory, problem solving, fluency, or the like), atrophy of parts of the brain, risk of future atrophy, medical diagnosis, physical condition, or the like. For example, the assessment 215 may determine that the patient has a high level of short-term and long-term memory, and is at an elevated risk for stroke and/or developing Alzheimer's disease at a certain age.

In an embodiment, each patient may receive a brain health score (or “score”) indicating a level of brain health during the assessment 215. The score may be calculated at least partially based on the patient information 205 and/or the test information 210 and may be modified as the patient progresses through a brain health program, for instance, to indicate improved brain health. According to some embodiments, the score may have multiple components, such as risk component (e.g., a dementia component, an Alzheimer's disease component), a fitness component (e.g., configured to indicate the mental and/or physical activity level of the patient), or the like.

The assessment 215 may be used to determine which type of treatment may be used to improve and/or maintain the brain health of the patient. The treatments may be formulated in a comprehensive personalized program 220 configured for the patient. The personalized program 220 may include a plurality of interventions 225a-225n, schedules for the interventions and a schedule for the overall program. The interventions 225a-225n may include any type of exercise, exam, test, function, activity, or the like that may be used to improve and/or maintain the brain health of a patient and the particular brain conditions thereof, such as memory, fluency, problem solving, or the risk of certain diseases. The schedules may be configured to provide personalized programs 220 that operate to most efficiently and effectively use the interventions 225a-225n to improve patient brain health and to monitor and assess the patient so that the patient may be performing the best interventions for his particular brain health at a particular point in time.

The interventions 225a-225n may include, without limitation, brain exercises, sleep optimization, stress reduction, a brain health optimization diet, physical exercise, brain coaching, cognitive stimulation, neurofeedback training, counseling, or the like. Brain coaching may include patient education about interventions to improve memory, diet, stress level, sleep, physical fitness, or the like, as well as instructions for improving memory skills. Brain coaching may be implemented by various individuals (for example, clinical psychologists) and/or systems (for example, automated learning programs). The brain coaching sessions may be performed over various durations at various intervals based on the assessment 215. For instance, brain coaching may be carried out one session a month for 60-90 minutes, one session a week for 60-90 minutes, or the like. EEG-Neurofeedback training may be used to teach patients how to optimize their brain function and performance based on feedback information obtained from an EEG brain activity readings while the patient is performing mental tasks.

The personalized program 220 is unique to the patient and his brain health, particularly the individual set of brain conditions that he is experiencing. In addition, the personalized program 220 is comprehensive in that it is configured to improve and/or maintain a plurality of brain conditions simultaneously or substantially simultaneously. The duration and/or frequency of the personalized program 220 and/or interventions 225a-225n may be configured based on the assessment 215. For instance, a patient with low brain health may have a longer personalized program 220 with more frequent performance of the interventions 225a-225n.

The patient may be monitored 230 at various intervals, such as after every intervention, every day, every week, every three months or every six months. According to some embodiments, during performance of the personalized program 220 the patient may be monitored 230 by the same practitioners, personnel and/or electronic monitoring (including the use of software applications configured to monitor patients on a personalized program) and/or at the same healthcare clinic to facilitate consistent treatment and efficient use of knowledge about the patient and her personalized program.

According to some embodiments, portions of the patient information 205 and/or test information 210 may be determined again and used to generate, and the information used to generate a new assessment 215. The personalized program 220 and the interventions 225a-225n may be modified based on the new assessment 215. For example, if the patient demonstrates improvement in memory skills and no improvement in problem solving skills, interventions 225a-225n directed toward improving memory skills may be reduced and/or eliminated and interventions directed toward improving problem solving skills may be added and/or increased.

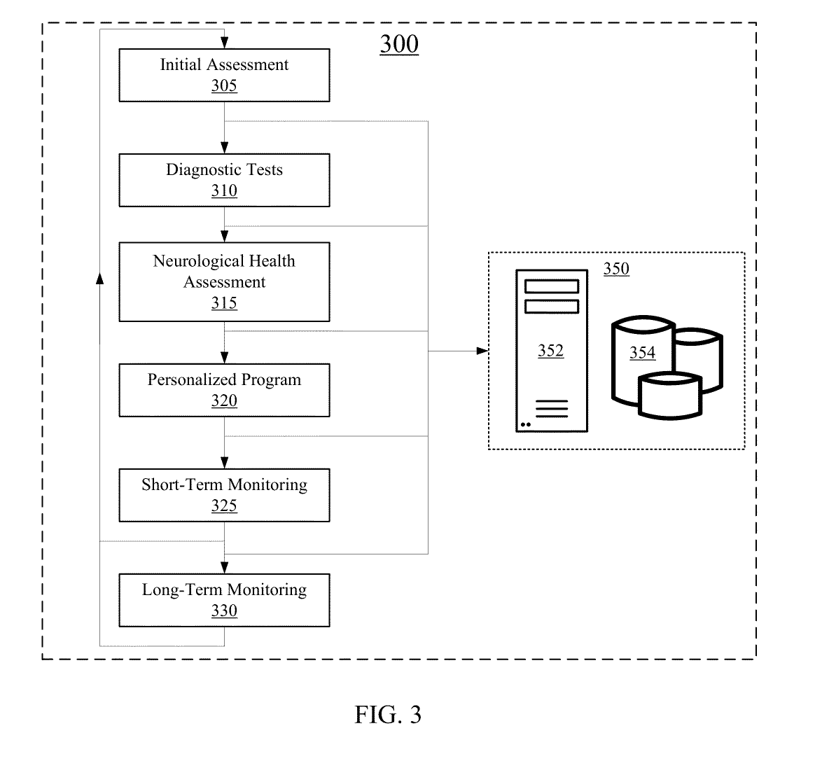

FIG. 3 depicts an illustrative system for generating a brain health program according to some embodiments. As shown in FIG. 3, the system may include an initial assessment 305 that may be generated based on information from the patient, such as medical records, answers to questionnaire and/or interview questions. The initial assessment 305 may be used to determine a set of diagnostic tests 310, for instance, that may be useful in determining the brain health of the patient. For example, it may be determined from the initial assessment 305 that the patient has a low activity level, has had a stroke, is having memory problems, and/or has a family history of dementia. As such, the system 300 may determine that one or more diagnostic tests 310 configured to measure, quantify or visualize the brain conditions indicated by the initial assessment 305, such as an MRI, a CPET, or the like.

The information obtained from the diagnostic tests 310, in combination with the information obtained from the initial assessment 305, may be used to generate a brain health assessment 315. The brain health assessment 315 may include a comprehensive picture of the brain health of the patient including, without limitation, brain strengths and weaknesses, desired areas of improvement, patient concerns, current health issues (for instance, hypertension, stroke, concussion, or the like), risks of potential health issues, or the like. The system 300 may generate a personalized program 320 for the patient based on the brain health assessment 315, including various interventions and/or schedules as described herein.

The patient may perform the personalized program 320, with assistance from various medical personnel, such as doctors, psychologists, brain coaches, or the like. During performance of the personalized program 320, information associated with the patient may be monitored on a short-term basis through a short-term monitoring 325 process. For instance, the patient may answer questions and/or perform medical tests at predetermined intervals, such as every week, every month or the like. Long-term monitoring 330 may be used to observe the progress and/or regression of the patient over a longer period of time, such as months or years. Information from short-term monitoring 325 and/or long-term monitoring 330 may be used to generate a new initial assessment 305, diagnostic testing 310, brain health assessment 315, and/or personalized program 320.

As shown in FIG. 3, the system 300 may include a network 350 including various logic devices 352, such as a server computing device, which may generally include a processor, a non-transitory memory or other storage device for housing programming instructions, data or information regarding one or more applications, and other hardware, including, for example, a central processing unit (CPU), read only memory (ROM), random access memory (RAM), communication ports, controller, and/or storage devices. The network 350 may also include a database 354. The network 300 may be a part of various information networks, such as a health information system (HIS), a picture archiving and communication system (PACS), or the like.

Information (“system information”) from the various system processes 305, 310, 315, 320, 325, 330 may be transmitted to the logic devices 352 and/or databases 354. The system information may be used by the system 300 and/or operators of the system for various purposes. For instance, the system information may be used as a historical information system. According to some embodiments, the system information may be used to generate one or more aspects of the system 300, such as an initial assessment 305, diagnostic testing 310, brain health assessment 315, and/or personalized program 320. For instance, the logic devices 352 may include an application (a “system application”) configured to analyze the system information to make certain determinations associated with generating an initial assessment 305, diagnostic testing 310, brain health assessment 315, and/or personalized program 320. For example, the system application may be configured to automatically determine which diagnostic test, interventions may be best for the patient based on their medical history, patient information, personal brain health goals, practitioner preferences, and/or diagnostic tests. In another example, the system application may use the system information to learn the effectiveness of the personalized programs 320 for the patients. This effectiveness learning may be used to educate practitioners operating the system 300 and/or to make future determinations regarding diagnostic tests, interventions and/or personalized programs for the same or different patients.

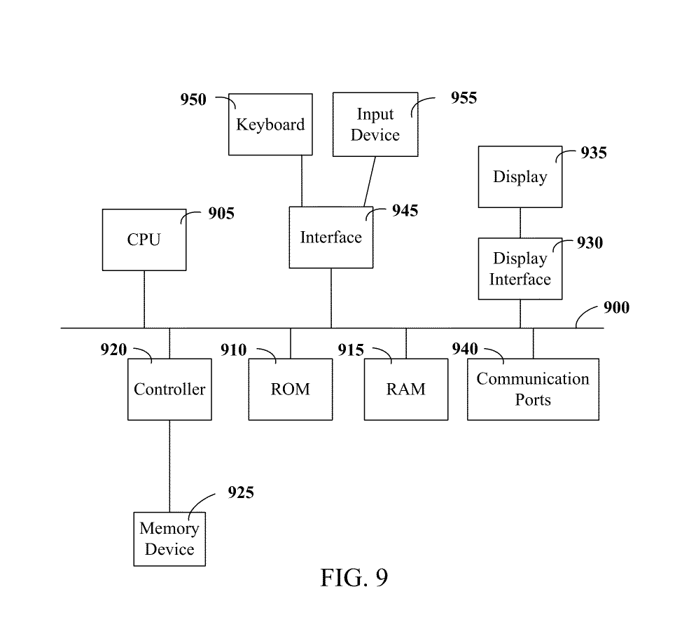

In another embodiment, an external client computing device (not shown) may be configured to access the network 350, such as a laptop computer, smartphone, personal digital assistant (PDA), tablet computing device, or any other logic and/or computing device now known or developed in the future (see FIG. 9 for a non-limiting example of a computing device). The client computing device may be capable of communicating with the system 300 and/or the system application, such as through a web application, a mobile application (a “mobile app” or “app”), a client application, the Internet, or the like. In this manner, a patient may transmit and/or receive information from the system 300 and/or the system application. For instance, the patient may be able to transmit information associated with their patient information, personal program 320, such as information associated with their performance of an intervention. In another instance, the patient may be able to access and view their information stored within the system 300 and/or system application.

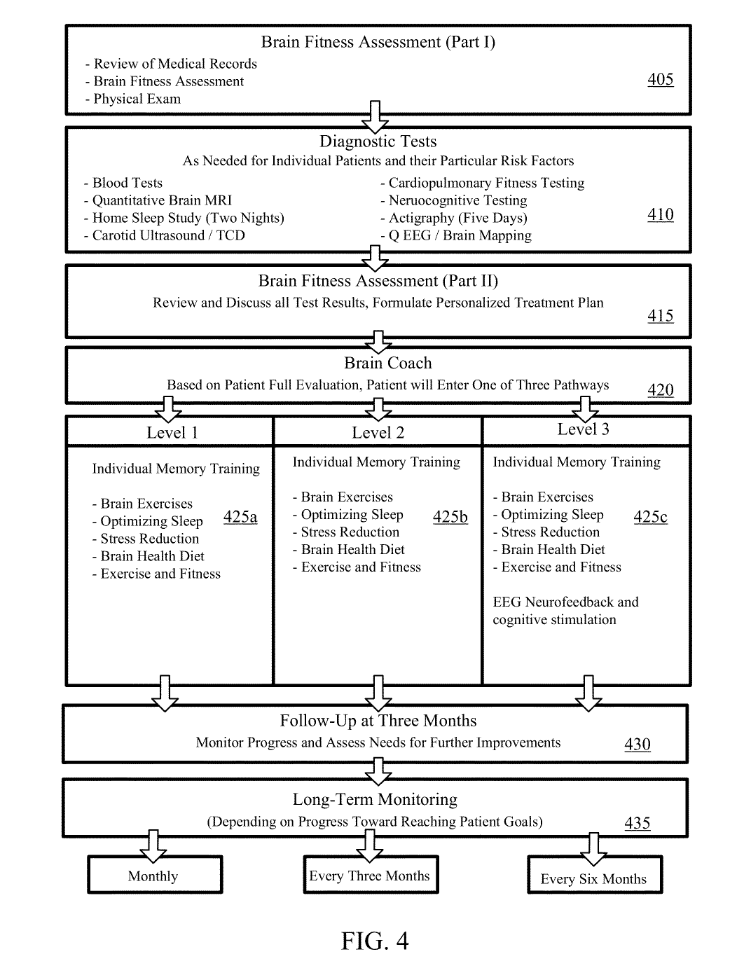

FIG. 4 depicts an illustrative method for generating a brain health program according to an embodiment. As shown in FIG. 4, the method may include an initial Brain Fitness Assessment 405 that may be used to determine which Diagnostic Tests 410 may be ordered for the patient. A Brain Fitness Assessment 415 may be generated for the patient that includes a personalized treatment plan. A Brain Coach 420 may be used to implement one of multiple potential Personalized Pathways 425a-425c that may be generated for the patient. The progress and/or regression of the patient may be monitored by various follow-up and/or monitoring processes, such as a Follow Up at 3 Months 430 and/or Long-Term Monitoring 435, which may consist of monthly, every three months, every six months, or the like.

Brain health programs configured according to some embodiments described herein provide multiple health and/or cognitive benefits to patients. Illustrative and non-restrictive examples of health and/or cognitive benefits include improved memory, problem solving skills, mental clarity, fluency, increased brain connections, and/or increases in the size of particular parts of the brain including, without limitation, the hippocampus.

An example study of brain health programs included an observational study measuring the effectiveness of three-month brain health programs individualized for thirty-four patients. The patients had various brain health characteristics, including a risk of Alzheimer's disease and an Mini Mental State Exam (MMSE) score greater than about 22. The brain health programs included various interventions, including interventions according to some embodiments described herein including neurofeedback, cognitive skills training, meditation, brain health diet counseling, nutritional supplements, addressing patient risk factors (for example, lack of exercise, poor sleep patterns, or the like), and helping patients increase their fitness level.

The example study included various baseline and post-program evaluations of patient brain and physical health including neurological exams, cognitive testing, CPET (for example, for VO₂), brain MRIs, blood tests, and quantitative EEGs (QEEG) (for example, to evaluate brain wave activity). After a baseline evaluation was completed, patients began a twelve-week program that consisted of at least two neurofeedback sessions, two cognitive skills training sessions, and one counseling/meditation session per week.

The example study demonstrated various improvements in the patients' brain health and fitness. For example, a comparison of the patients' baseline and post-program cognitive evaluations showed significant improvement in short term memory and improvement in visual learning, executive function and attention. There was also improvement in physical fitness, as measured by VO₂-max. An analysis of variance (ANOVA) on patients who completed all components of the brain health program demonstrated that there was significant improvement in immediate list learning and delayed visual learning. In general, a three-month, multidisciplinary brain health program that focuses on, among other things, neurofeedback, cognitive training, fitness training, and mediation may substantially improve memory, executive function, and increase the size of the hippocampus in patients, for example, with age associated cognitive decline.

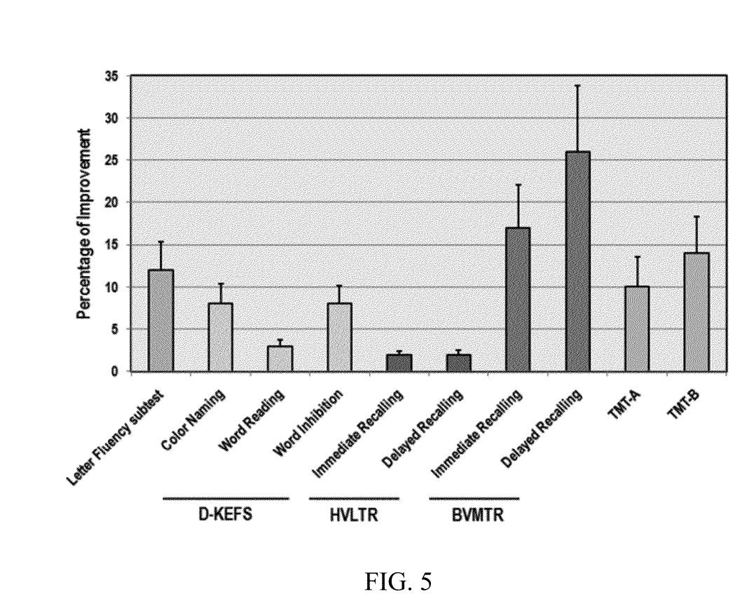

FIGS. 5-8 graphically depict various aspects of the example study. FIG. 5 depicts a percentage change in the metrics of the brain health program in the example study. For example, in FIG. 5, baseline and post-program performance was compared on different components of the brain health program. The battery of neuropsychological tests included at least: letter fluency, color naming, word reading, word inhibition, immediate verbal recalling, delayed verbal recalling, immediate visual recalling, delayed visual recalling, trail making test-A, and train making test-B. There was an overall improvement that was statistically significant (p<0.026). Of the people who improved, the most significant benefits were seen in letter fluency (12%), immediate visual recalling (17%), delayed visual recalling (26%), trail making test-A (10%), and train making test-B (14%).

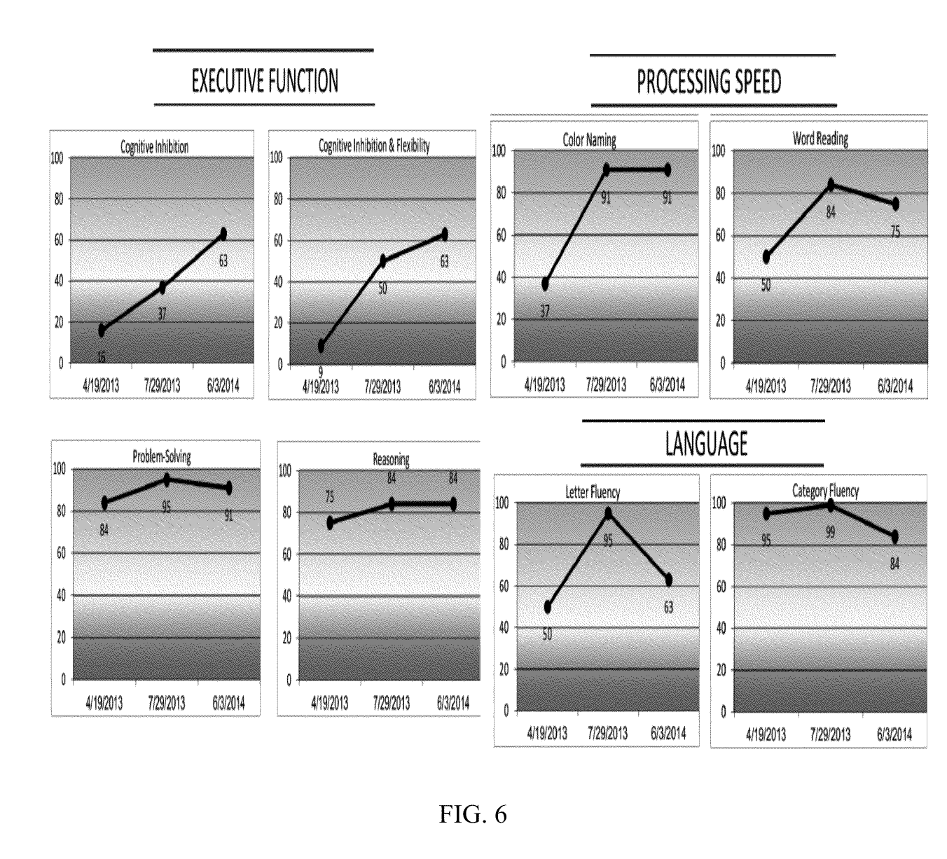

FIG. 6 depict the short term and sustained long-term benefits of the program for one patient. In this patient, significant improvement was seen in various tests of executive function (e.g., Stroop test, cognitive inhibition, cognitive flexibility, reasoning), processing speed (e.g., color naming, word reading), and language (e.g., category fluency and letter fluency). FIG. 6 also shows that the improvements seen at 3 months (second time point) were sustained after one year post-treatment.

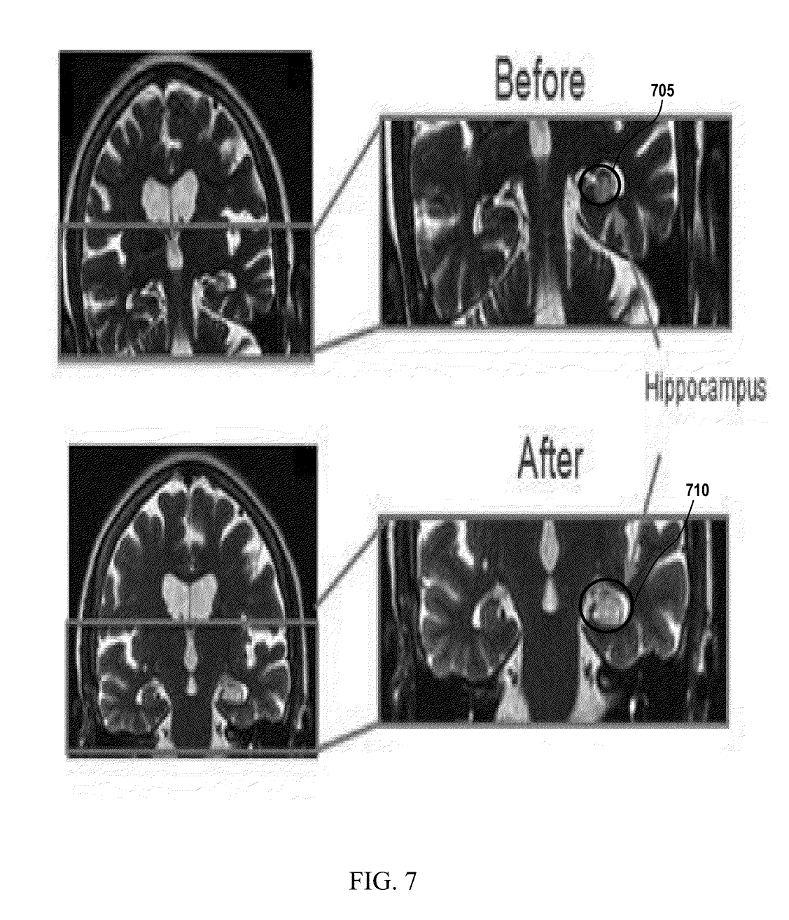

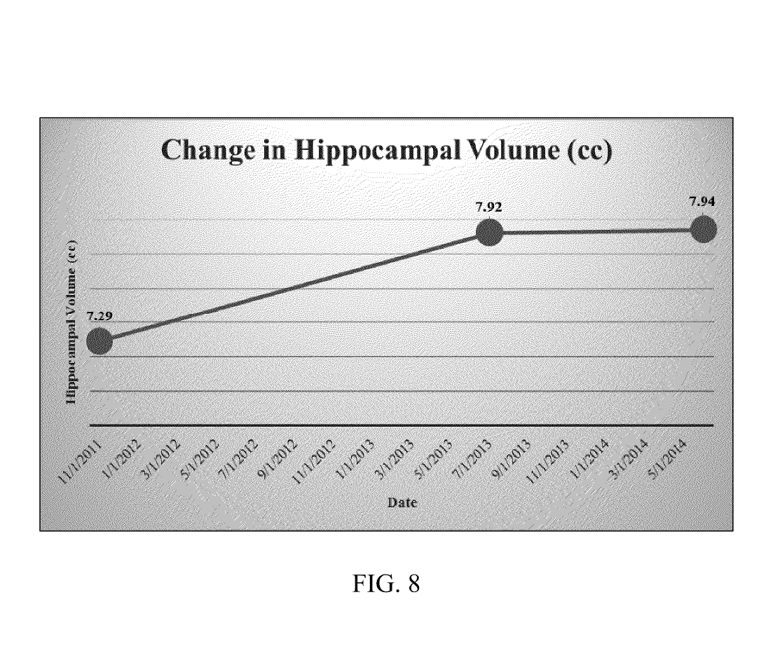

FIGS. 7 and 8 provide a comparison of hippocampal volume of a patient before and after completion of the brain fitness program in the example study. FIG. 7 depicts the visually detectable increase in the volume of the hippocampus as shown by MRI before treatment 705 and after 3 months of treatment 710. FIG. 8 shows the surprising result that the size of the hippocampus grew by about 8.6% (from 7.29 cm cube to 7.92 cm cube). Given that hippocampus shrinks by 0.5% per year, this is equivalent to being approximately 18 years younger in brain age. Another surprising result was that the improvement in the size of the hippocampus in this patient persisted over one year post-treatment, and the size of the hippocampus actually increased by another 0.9% (from 7.92 to 7.94 cm³). An increase in hippocampal volume was observed in other patients participating in the example study.

Accordingly, participation in a comprehensive and personalized brain health program generated according to embodiments provided herein may lead to improved brain health metrics, such as memory, problem solving, attention, executive function, or the like. In addition, participation in a comprehensive and personalized brain health program generated according to embodiments provided herein may lead to increased brain size (for example, increased hippocampal volume) and/or increased brain connections.

FIG. 9 depicts a block diagram of exemplary internal hardware that may be used to contain or implement the various computer processes and systems as discussed above. A bus 900 serves as the main information highway interconnecting the other illustrated components of the hardware. CPU 905 is the central processing unit of the system, performing calculations and logic operations required to execute a program. CPU 905, alone or in conjunction with one or more of the other elements disclosed in FIG. 9, is an exemplary processing device, computing device or processor as such terms are used within this disclosure. Read only memory (ROM) 530 and random access memory (RAM) 535 constitute exemplary memory devices.

A controller 920 interfaces with one or more optional memory devices 925 to the system bus 900. These memory devices 925 may include, for example, an external or internal DVD drive, a CD ROM drive, a hard drive, flash memory, a USB drive or the like. As indicated previously, these various drives and controllers are optional devices. Additionally, the memory devices 925 may be configured to include individual files for storing any software modules or instructions, auxiliary data, common files for storing groups of results or auxiliary, or one or more databases for storing the result information, auxiliary data, and related information as discussed above.

Program instructions, software or interactive modules for performing any of the functional steps associated with generating, implementing, developing, and/or administering brain health programs as described above may be stored in the ROM 930 and/or the RAM 935. Optionally, the program instructions may be stored on a tangible computer-readable medium such as a compact disk, a digital disk, flash memory, a memory card, a USB drive, an optical disc storage medium, such as a Blu-ray™ disc, and/or other recording medium.

An optional display interface 930 may permit information from the bus 900 to be displayed on the display 935 in audio, visual, graphic or alphanumeric format. Communication with external devices may occur using various communication ports 940. An exemplary communication port 940 may be attached to a communications network, such as the Internet or a local area network.

The hardware may also include an interface 945 which allows for receipt of data from input devices such as a keyboard 950 or other input device 955 such as a mouse, a joystick, a touch screen, a remote control, a pointing device, a video input device and/or an audio input device.

It will be appreciated that various of the above-disclosed and other features and functions, or alternatives thereof, may be desirably combined into many other different systems or applications. It will also be appreciated that various presently unforeseen or unanticipated alternatives, modifications, variations or improvements therein may be subsequently made by those skilled in the art which alternatives, variations and improvements are also intended to be encompassed by the following claims.

What is claimed is:

1. A method for generating at least one comprehensive and personalized brain health program for a patient, the method comprising:
receiving health information from the patient;
receiving diagnostic testing information associated with the patient;
generating a brain health assessment for the patient based on at least a portion of the health information and the diagnostic testing information, the brain health assessment comprising a plurality of brain health conditions; and
generating the at least one comprehensive and personalized brain health program for the patient based on the at least one assessment, the at least one comprehensive and personalized brain health program comprising a plurality of interventions configured to improve or maintain the plurality of brain health conditions.

2. The method of claim 1, further comprising receiving updated patient information and updated diagnostic testing information at a predetermined interval after the patient has started the comprehensive and personalized brain health program; and
generating an updated assessment based on the updated patient information and updated diagnostic testing information.

3. The method of claim 3, further comprising generating at least one updated comprehensive and personalized brain health program for the patient based on the at least one updated assessment.

4. The method of claim 1, wherein the patient information comprises information from at least one of the following: patient medical records, a cognitive screening, a neurological exam, a physical exam, and a brain fitness assessment.

5. The method of claim 1, wherein the diagnostic testing information comprises information from at least one of the following: a blood test, a brain magnetic resonance imaging exam, a quantitative brain magnetic resonance imaging exam, a sleep study, a carotid ultrasound, a transcranial Doppler, a cardioupulmonary fitness test, a neurocognitive test, an actigraph, an electroencephalography, and a quantitative electroencephalography.

6. The method of claim 1, wherein the plurality of brain health conditions comprise two or more of the following: a neurological disease, reduced cerebral blood flow, hippocampal atrophy, cortical atrophy, lacunar stroke, white matter abnormalities, memory conditions, and cognitive conditions.

7. The method of claim 6, wherein the neurological disease comprises at least one of Alzheimer's disease, mild cognitive impairment, and Parkinson's disease.

8. The method of claim 1, wherein the plurality of interventions comprises at least two or more of the following: brain exercises, sleep optimization, stress reduction, brain health diet, physical exercise, electroencephalography-based neurofeedback, counseling, and cognitive stimulation.

9. The method of claim 1, wherein to improve or maintain the plurality of brain health conditions comprises causing an increase hippocampal volume.

10. The method of claim 9, wherein the increase in hippocampal volume is sustained for at least one year.

11. A system for generating at least one comprehensive and personalized brain health program for a patient, the system comprising:
a processor; and
a non-transitory, computer-readable storage medium in operable communication with the processor, wherein the computer-readable storage medium contains one or more programming instructions that, when executed, cause the processor to:
receive health information associated with the patient;
receive diagnostic testing information associated with the patient;
generate a brain health assessment for the patient based on at least a portion of the health information and the diagnostic testing information, the brain health assessment comprising a plurality of brain health conditions; and
generate the at least one comprehensive and personalized brain health program for the patient based on the at least one assessment, the at least one comprehensive and personalized brain health program comprising a plurality of interventions configured to improve or maintain the plurality of brain health conditions.

12. The system of claim 9, wherein the computer-readable storage medium further contains one or more programming instructions that, when executed, cause the processor to receive updated patient information and updated diagnostic testing information at a predetermined interval after the patient has started the comprehensive and personalized brain health program; and
generate an updated assessment based on the updated patient information and updated diagnostic testing information.

13. The system of claim 10, wherein the computer-readable storage medium further contains one or more programming instructions that, when executed, cause the processor to generate at least one updated comprehensive and personalized brain health program for the patient based on the at least one updated assessment.

14. The system of claim 9, wherein the patient information comprises information from at least one of the following: patient medical records, a cognitive screening, a neurological exam, a physical exam, and a brain fitness assessment.

15. The system of claim 9, wherein the diagnostic testing information comprises information from at least one of the following: a blood test, a brain magnetic resonance imaging exam, a quantitative brain magnetic resonance imaging exam, a sleep study, a carotid ultrasound, a transcranial Doppler, a cardiopulmonary fitness test, a neurocognitive test, an actigraph, an electroencephalography, and a quantitative electroencephalography.

16. The system of claim 9, wherein the plurality of brain health conditions comprise two or more of the following: a brain disease, reduced cerebral blood flow, hippocampal atrophy, cortical atrophy, lacunar stroke, white matter abnormalities, memory conditions, and cognitive conditions.

17. The system of claim 14, wherein the brain disease comprises at least one of Alzheimer's disease and Parkinson's disease.

18. The system of claim 9, wherein the plurality of interventions comprises at least two or more of the following: brain exercises, sleep optimization, stress reduction, brain health diet, physical exercise, electroencephalography-based neurofeedback, counseling, and cognitive stimulation.

19. The system of claim 1, wherein to improve or maintain the plurality of brain health conditions comprises causing an increase hippocampal volume.

20. A computer-readable storage medium having computer-readable program code configured to generate at least one comprehensive and personalized brain health program embodied therewith, the computer-readable program code comprising:
computer-readable program code configured to receive health information associated with the patient;
computer-readable program code configured to receive diagnostic testing information associated with the patient;
computer-readable program code configured to generate a brain health assessment for the patient based on at least a portion of the health information and the diagnostic testing information, the brain health assessment comprising a plurality of brain health conditions; and
computer-readable program code configured to generate the at least one comprehensive and personalized brain health program for the patient based on the at least one assessment, the at least one comprehensive and personalized brain health program comprising a plurality of interventions configured to improve or maintain the plurality of brain health conditions.
